(19) United States
(12) Patent Application Publication
(10) Pub. No.: US 2006/0287347 A1
(43) Pub. Date: Dec. 21, 2006

(54) Methods for treating smooth muscle disorders using trospium

(76) Inventor: A.K. Gunnar Aberg, Sarasota, FL (US)

Correspondence Address: NIELDS & LEMACK, 176 EAST MAIN STREET, SUITE 7, WESTBORO, MA, 01581, US

(21) Appl. No.: 11/453,748

(22) Filed: Jun. 15, 2006

Related U.S. Application Data
(60) Provisional application No. 60/692,471, filed on Jun. 21, 2005.

(51) Int. Cl. A61K 31/4747

(52) U.S. Cl. 514/278

(57) ABSTRACT

Methods are disclosed using Trospium Chloride, an antimuscarinic smooth muscle relaxant, for the treatment of urinary incontinence, and other disorders, while avoiding the concomitant liability of adverse side effects associated with conventional antimuscarinic treatment.

This application claims priority of Provisional Application Ser. No. 60/692,471 filed Jun. 21, 2005, the disclosure of which is hereby incorporated in its entirety by reference.

### FIELD OF THE INVENTION

This invention relates to a compound named trospium, the chloride salt thereof having the formula:

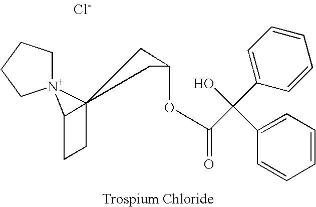

The generic name Trospium Chloride (CAS-10405-02-4; INN) refers to an anticholinergic compound with the chemical name endo-3-[(hydroxydiphenyl-acetyl)oxy]spiro[8-azoniabicyclo[3.2.1]octane-8,1′-pyrrolidinium]chloride (other chemical names exist); C₂₅H₃₀ClNO₃; MW=427.97.

Trospium can be synthesized as described by Pfleger R. et al. in U.S. Pat. No. 3,480,626 and by Bertholdt H. et al. in Arzneimittel-Forsch, 1967, 17: 719-726.

Trospium Chloride may be purchased from Galen Ltd, Craigavon, UK or from Madaus AG, Koln, Germany. The compound trospium can also be extracted from 20 mg Trospium Chloride tablets (Regurin®, Madaus in Germany or Sactura®, Indevus in the USA), using extraction methods commonly known to those skilled in the art.

Trospium has several known metabolites, the most well known being the spiroalcohol. The spiroalcohol metabolite has antimuscarinic activities that are believed to contribute to the therapeutic activity of Trospium Chloride.

The present invention relates specifically to the therapeutic use of trospium, possible prodrugs thereof and the active metabolites of trospium and the possible prodrugs thereof, and pharmaceutical compositions containing at least one of said compounds for treating smooth muscle hyperactivity disorders, such as for example urinary disorders, including urinary incontinence and pollakiuria, and gastrointestinal disorders, including gastrointestinal hyperactivity, and other smooth muscle hyperactivity or hyperreactivity disorders including those occurring in conjunction with asthma, bronchitis, urolithiasis, cholelithiasis and choledocholithiasis, while avoiding certain serious cognitive side effects, said cognitive side effects being considered to be hallmarks of anticholinergic medication.

The risk for severe cognitive side effects of antimuscarinic medication is particularly high in patients with pre-existing cognitive disorders or in patients at risk for developing cognitive disorders or in patients already using other medication that may have effects on cognition, such as for example oxybutynin (Ditropan®, Alza), or in elderly patients. In this document, the term “elderly” refers to individuals at the age of 65 years and older.

### BACKGROUND OF THE INVENTION

Trospium has been shown to reduce bladder hyperactivity in patients suffering from urinary incontinence and exerts spasmolytic effects on the bladder by inhibiting the effects of acetylcholine on smooth muscle. Thus, Trospium Chloride is an anticholinergic drug. Trospium Chloride has selectivity for muscarinic receptors over nicotinic receptors and as a result, no blocking effects are observed at skeletal neuromuscular junctions. Thus, the anticholinergic drug Trospium Chloride can also be called an antimuscarinic drug. Active metabolites of Trospium Chloride exert antimuscarinic activities that are believed to account for part of the therapeutic activity of trospium.

The terms ‘anticholinergic’ and ‘antimuscarinic’ are interchangeable in this document.

The terms “predisposed” to a cognitive disorder and “propensity” for developing cognitive disorders, which are interchangeably used herein, refer to humans who are at risk for developing Cognitive Disorders, such as for example elderly patients or patients suffering from certain diseases, such as for example AIDS or cancer or type-II diabetes or patients taking medication that can cause cognitive disorders, such as for example oxybutynin. Individuals who are predisposed to conductive disorder may suffer from intermittent or “on and off” symptoms of cognitive disorders. After being diagnosed, cognitive disorders may also be intermittent, or appear “on-and-off” in patients; thus patients suffering from cognitive disorders may have symptoms that alternate between more or less severe.

The term “urge incontinence” in this document includes “over-active bladder” (OAB) and the disease called pollakiuria (frequent urinations).

Cognition is the ability of the brain to think, which includes the ability to process and store processes information and to solve problems. Other definitions of the term “cognition” exist and the present invention is not limited to one particular specific definition. Most definitions make reference to intellectual processes in the human brain that involve thinking or the acquirement of knowledge. As known to those skilled in the art of biology, cognition is a high level of behavior and is considered to be exclusive and unique to humans. The terms “Cognitive Disorders” and “cognitive disorder” refer to disorders in a human, where a limitation of cognitive functioning is the main feature.

There are various cognitive disorders. Cognitive Disorder NOS (Cognitive Disorders Not Otherwise Specified) is a term that embraces various types of Cognitive Disorders. Diagnosis of Cognitive Disorder NOS is done when a patient has a syndrome of cognitive impairment that does not meet the criteria for delirium and certain other disorders as known to those skilled in the art of diagnosing psychiatric diseases.

The present invention deals with the treatment of urinary incontinence in patients suffering from cognitive disorders and those at risk for developing cognitive disorders.

Individuals at risk for developing cognitive diseases are said to be predisposed to cognitive disorders and include for example elderly patients and patients suffering from certain diseases, such as for example atherosclerosis, multiple sclerosis, Parkinson's disease, Pick's disease, viral or bacterial encephalitis, Lewy body disease, subdural hematoma, brain tumor, AIDS, cancer and diabetes. Abnormalities of a patient's metabolism or hormones may also be causative in the development of cognition disorders and individual with such abnormalities are considered as being predisposed for cognitive disorders; examples are hypo- or hyperthyroidism, steroid abuse, deficiency or low body levels of vitamin B12, B1, or B3, chronic alcohol abuse or illegal drug abuse. A causative factor for the development of cognitive disorders may also be one or more of the medications that the patient is taking. It is believed that hereditary factors may predispose an individual for cognitive disorders that may develop later in life. It is also believed that obesity constitutes a significantly increased risk for the development of cognitive disorders. Individuals who are predisposed to cognitive disorder may complain of intermittent or “on and off” symptoms of cognition disorders.

Early symptoms of cognition disorders that can be observed are for example difficulties in reasoning and accepting changes, becoming confused over time, place and/or direction, expressing impaired judgment, showing changes in personality, suffering from urinary incontinence and/or is becoming passive and losing initiative. These patients are no longer considered as being predisposed to cognitive disorders, but are considered to be individuals who suffer from the disease. Symptoms at the middle stage may include loss of certain cognitive abilities, such as the ability to learn, judge and reason, patient becoming emotionally unstable, patient needing help with the daily life, often experiencing urinary incontinence and/or confusing day and night. Symptoms at later stage include for example severe loss of cognitive abilities, neglecting personal hygiene, advanced incontinence, gradually losing weight and/or walking unsteadily and becoming confined to bed.

Other types of cognitive disorders are for example Huntington's disease, which leads to different Cognitive Disorders than for example Alzheimer's disease. Most patients suffering from Huntington's disease will at some stage be faced with the problem of urinary incontinence (Huntington's Disease Advocacy Center. http//www.hdac.org.) Mental Retardation is still another type of Cognitive Disorder, which occurs in young individuals and leads to severely reduced intelligence (IQ). Patients with mental retardation often suffer from fecal as well as urinary incontinence.

In addition to senile dementia with cognitive disabilities, Cognitive Disorders NOS include but are not limited to disorders such as Aphasia (breakdown of language skills, such as for example Anomia. Paraphasia, Agrammatism), Apraxia (deterioration of the ability to translate a verbal command into its motor expression), Amnesia, Agnosia (inability to associate an object with its meaning, to recognize objects and him/herself) or Executive Dysfunction (deterioration of ability to make goals or carry out plans to reach goals). Thus, cognitive disabilities can be a symptom of any of the aforementioned cognitive disorders. Cognitive deterioration may be caused by decreased concentration of the transmitter substance acetylcholine in forebrain cholinergic neurons that are for example localized in the nucleus basalis magnocellularis, which is the major source of cholinergic innervation to the neocortex and to the amygdala. Cognitive Disorders may also be caused by antimuscarinic effects in the septum-banda diagonalis complex, which is known to provide cholinergic innervation of the hippocampus. Numerous studies have revealed that dysfunction in these cholinergic neurons leads to cognitive deterioration. Senile dementia is caused by brain tissues being damaged and the functioning being diminished. Senile dementia is expressed as cognitive disorders and/or memory disorders depending on which brain tissues are deteriorating.

People who suffer from Cognitive Disorders may in serious cases fall in and out of consciousness and in all cases have problems with intellectual functions, such as for example attention, thinking, awareness, emotion, sleeping and waking.

Cognitive Disorders are often coupled to a specific medical condition (such as for example cancer, AIDS, type-II diabetes) and/or a pharmacological reaction to medication (such as for example anti-Parkinson, antispasmodic, antimigraine, antidiarrheal, antihistaminic, antiulcer, antidepressant, antipsychotic or bronchodilator medications) or are part of the ageing process in the brains of many patients.

The clinical diagnosis of Cognitive Disorder is usually made by a physician, who can use a Mini-Mental State Examination (MMSE) to measure the cognitive status of the patient. The MMSE shall only be performed by qualified medical personnel, and can be purchased by physicians from PAR Customer Support, 16204 N. Florida Avenue, Lutz, Fla. 33549. Similar tests for cognitive disorders exist and can be used instead of or together with the MMSE. Furthermore, experienced physicians may be able to diagnose cognitive disorders without the help of a standardized test.

It is a method of the present invention to determine if patients, who are suffering from smooth muscle disorders, are simultaneously suffering from or having a propensity for cognitive disorders and if said determination is positive, administering to said patients a therapeutically effective amount of trospium or an active metabolite thereof or a pharmaceutically acceptable salt thereof. Said determinations can be performed by a physician using interviews, physical examination and/or application of a standardized test, such as for example the Mini-Mental State Examination (MMSE).

To have a direct effect on certain areas of the brain, drugs have to cross the blood-brain barrier (BBB). This barrier is made up of layers of cells, surrounding the small blood vessels that supply the brain. The BBB was previously believed to be a simple mechanical barrier, keeping all large molecules and all charged molecule out of the brain. Thus, factors believed to influence the ability of drugs to cross the blood-brain barrier were believed to include ionization (pKa), lipophilicity (logP) and molecular size (MW<400). However, the knowledge about the nature of the BBB, its functions and its limitations have improved tremendously and we now know that the BBB is not simply a mechanical barrier, but also has mechanisms in place for the active transport of molecules out of the brain. The BBB can become “leaky” (more permeable) of numerous reasons, such as for example by influence from the hormone epinephrine that is known to cause leakage of the BBB. Diseases, such as cognitive disorders, type-II diabetes and hypertension are often correlated with increased permeability of the BBB. It has been suggested that at least some forms of Cognitive Disorders are caused by increased permeability of the blood brain barrier (Wardlaw J M, Sandercock P A G, Dennis M S, Starr J. Stroke, 2003, 34: 806.) It is also known that certain drugs can induce increased permeability of the BBB. Examples of such drugs are certain angiotensin converting enzyme inhibitors and phenylephrine. It is also well-known that the blood-brain barrier deteriorates with age (Toornvliet J R, van Berckel B N M, Luurtsema G, Lubberink M, Geldof A A, Bosch T M, Lammertsma A A, Franssen R J F. J Cerebral Blood Flow $ Metabolism, 2005, 25: 273; Bronge L, Wahlund L O. Dement Geriatr Cogn Disord, 2000, 11:263-267.) This is of importance in connection with the pharmacology of drugs for geriatric diseases like urinary urge incontinence that almost exclusively affects the elderly. Studies on blood-brain barrier being performed in healthy young volunteers are of no or very limited relevance to the elderly patients who suffer from urinary urge incontinence, particularly if the study do not include continuous drug administration over time (Todorova A, Vonderheid-Guth B, Dimpfel W. J Clin Pharmacol. 2001; 41:636-644), since age and cognitive disorders are often coupled to increased permeability of the BBB as pointed out above. In general, quaternary amines penetrate the healthy BBB less readily than tertiary amines, however, quaternary amines are known to cross the BBB, even in healthy individuals. Thus, as a well known example, the quaternary choline esterase inhibitor pyridostigmine was able to penetrate the BBB in American soldiers, causing the Persian Gulf Syndrome (PGS) in gulf war veterans. The Persian Gulf Syndrome includes effects on cognition (Haley R. W. & Kurt T. L. JAMA,. 1977: 231-239). Numerous other quaternary compounds are known to cross the BBB and cause CNS-mediated side effects, such as for example the quaternary drugs edrophonium that causes convulsions and restlessness and neostigmine that causes dizziness, convulsions, drowsiness and headache.

Furthermore, the BBB is absent from significant parts of the brain, such as for example the hypothalamus, the pituitary and pineal areas, area postrema and areas of the choroids plexus. Drugs can cause cognitive side effects on the hypothalamic-pituitary axes, as has been described for example for benzodiazepines, of which diazepam (Valium®) is an example.

It can be assumed that quaternary compounds, such as trospium will be able to cross the blood-brain barrier rather slowly in healthy and young individuals, but in the elderly individuals and particularly in elderly patients with existing diseases, quaternary compounds, do have the ability to cross the blood-brain barrier. The reason why trospium does not have negative effects on cognition, although quaternary molecule can enter into the brain of the patients using this type of medications, is unknown.

To our knowledge, no known reference teaches or enables the methods of the present invention comprising administering trospium to a human suffering from Cognition Disorders or being at risk for developing Cognition Disorders; nor do the published references alone or in combination suggest these methods. It is of importance to note that patients in earlier stages of Cognition Disorders, many patients express cognitive malfunctions that have a tendency to be intermittent or “come and go”. Elderly patients are always considered to be at risk for developing cognitive disorders of various types, including Cognitive Disorders and Cognitive Disorders NOS.

### SUMMARY OF THE INVENTION

Pharmacological studies of Trospium Chloride have now been performed in comparison with known and marketed antimuscarinic drugs with therapeutic activity against cholinergically mediated diseases, such as for example urinary incontinence.

The present studies have confirmed that trospium, as well as darifenacin, tolterodine and oxybutynin, have potent antimuscarinic activity.

It has now been found that while antimuscarinic drugs for incontinence, such as for example oxybutynin, can cause cognitive side effects, trospium, surprisingly, does not cause this side effect, even under circumstances where the permeability of the blood-brain barrier has experimentally been increased. Thus, trospium can safely be used as a treatment for urinary urge incontinence and other smooth muscle disorders while not aggravating cognitive disorders, in patients suffering from such disorders or in patients being predisposed for cognitive disorders, which is contrary to other anticholinergic medications that are used to treat said smooth muscle diseases.

Since the ability of cognitive thinking is the major difference between humans and other animal species, it is not possible to find a perfect animal model for cognitive disorders. However, it is well known by those skilled in the arts of pharmacology and toxicology that certain animal models, such as for example impairment of learning behavior by drugs in laboratory animals, is highly indicative of risk for cognitive side effects of said drugs in humans. It has now surprisingly been found that trospium does not cause said type of learning impairment in laboratory animals and it is therefore concluded that trospium will offer anticholinergic treatment for muscarinic disorders, including urinary voiding disorders such as urinary urge incontinence and for gastrointestinal disorders, including gastric motility disorders such as diarrhea in humans, while avoiding the concomitant liability of cognitive adverse side effects associated with currently used antimuscarinic drugs. Trospium will therefore offer potential for anticholinergic treatment for smooth muscle disorders, including urinary incontinence and gastrointestinal smooth muscle disorders including increased activity or hyperactivity of said gastrointestinal smooth muscle and kidney and gall bladder disorders, such as urolithiasis and cholelithiasis and choledocholithiasis, while being devoid of cognitive side effects and while not causing worsening of cognitive disorders of patients suffering from such disorders and being devoid of causing cognitive disorders in patients at risk for expressing the symptoms of cognitive disorders, said symptoms of cognitive disorders being known as stop-and-go symptoms in many patients, as known by those skilled in the art.

In cases where urgent treatment is preferred, trospium can be administered parenterally, such as by the intravenous route to rapidly alleviate smooth muscle spasm and the pain in connection with urolithiasis or cholelithiasis or choledocholithiasis. Continued treatment of the patients can use oral administration of trospium.

Urinary incontinence is a disease that in many patients is not consistent, but it is recurrent and patients can take their medication to prevent the reoccurrence of the symptoms of their disease. The magnitude of a prophylactic or therapeutic dose of a compound of the present invention in the acute or chronic management of disease will vary with the severity and nature of the condition to be treated and with the route of administration. The dose and the frequency of the dosing will also vary according to the age, body weight, expected results and response of the individual patient. Doses as low as 1 mg to as high as 240 mg, as single dose or divided into repeated doses, may be administered daily to patients in need of such therapy. More preferred are daily doses of 10 mg to 60 mg of trospium chloride, as a single daily dose or divided into repeated doses during a 24-hour period. Most often, the daily oral dose of Trospium Chloride is one 20-mg tablet twice daily to patients suffering from urinary urge incontinence. In managing the patient, the therapy may be initiated at a lower dose, perhaps at about 5 mg to about 10 mg, twice daily, and is usually increased up to 20 mg depending on the patient's global response. It may be necessary to use dosages outside these ranges in some cases, as will be apparent to those skilled in the art. Further, it is noted that the clinician or treating physician will know how and when to interrupt, adjust, or terminate therapy in conjunction with individual patient response. It is obvious for those skilled in the art that controlled-release formulation of trospium will constitute advantageous dosage forms that will not only offer convenience, but will also avoid or decrease acute antimuscarinic side effects, such as dry mouth and blurry vision, which are coupled to peak plasma concentrations of the drug.

The terms “a therapeutically effective amount” and “an amount sufficient to treat the disorder but insufficient to cause adverse effects” are encompassed by the above-described dosage amounts and dose frequency schedule.

Any suitable route of administration may be employed for providing the patient with an effective dosage of the compounds of the present invention. For example, oral, sublingual, parental (i.e. subcutaneous, intramuscular, intravenous, etc.), transdermal, vaginal, aerosol and like forms of administration may be employed. Additionally, the drug may be administered directly into the bladder, as described for oxybutynin by Massad C. A., et al. in J. Urol. 148, 595-597 (1992) and for Trospium Chloride by Schwantes U., et al. in U.S. Pat. No. 5,998,430 or rectally directly into the gastrointestinal canal. Dosage forms include tablets, troches, dispersions, suspensions, solutions, capsules, suppositories, microencapsulated systems, slow-release and controlled release systems, transdermal delivery systems, and the like.

Prodrugs of trospium or prodrugs of the spiroalcohol metabolite can be prepared by those skilled in the art, as has been described for an active metabolite of tolterodine by Sparf B. et al. in EP 0957 073 Al, and may be administered in accordance with the present invention.

The pharmaceutical compositions of the present invention comprise trospium or a metabolite thereof as the active ingredient, or any pharmaceutically acceptable salt thereof, and may also contain a pharmaceutically acceptable carrier, and optionally, other therapeutic ingredients.

The terms “pharmaceutically acceptable salts” or “pharmaceutically acceptable salt thereof” refer to possible salts of trospium and the active metabolite of trospium. Said salts of trospium may be prepared from pharmaceutically acceptable non-toxic acids. Suitable pharmaceutically acceptable salts for the compounds of the present invention include the chloride of trospium, but also other halogen salts and salts such as acetic, benzenesulfonic (besylate), benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, p-toluenesulfonic, succinic, sulfuric, tartaric, and the like, if possible to synthesize and if pharmaceutically acceptable. Particularly preferred is Trospium Chloride.

The compositions of the present invention include suspensions, solutions, elixirs or solid or semi-solid dosage forms. Carriers such as starches, sugars, and microcrystalline cellulose, diluents, granulating agents, lubricants, binders, disintegrating agents, and the like are suitable in the case of oral solid preparations (such as powders, capsules, and tablets), and oral solid preparations are preferred over the oral liquid preparations. Because of their ease of administration, tablets and capsules represent the more advantageous oral dosage unit forms, in which case solid or semi-solid pharmaceutical carriers are employed. If desired, tablets may be coated, using standard aqueous or nonaqueous techniques. Capsules can be hard or soft.

In addition to the common dosage forms set out above, the compounds of the present invention may also be administered by controlled release means and delivery devices to obtain improved pharmacokinetic profiles (such as sustained and stable plasma levels or prolonged duration of activity) or decreased side effects. Thus, controlled-release tablet formulations of Trospium Chloride, allowing once-daily administration, have obvious advantages for the patient, as realized by those skilled in the art.

Pharmaceutical compositions of the present invention, suitable for oral administration, may be presented as discrete unit dosage forms such as capsules, cachets, suppositories, or tablets, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the active ingredient with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made by molding, in a suitable machine, a mixture of the powdered compound moistened with an inert liquid diluent. All of the foregoing techniques are well know to persons of skill in the pharmaceutical art. Each tablet or capsule may contain from about 1 mg to about 60 mg of the active ingredient. An example of an oral unit dose formulation is shown below.

### EXAMPLE 1

Oral Unit Dosage Formulation (Tablets)

| | | per batch of | |
|---|---|---|---|
| Ingredients | per tablet | 10,000 tablets | |
| Trospium Chloride | 20 mg | 200 | g |
| Microcrystalline | 30 mg | 300 | g |
| cellulose | | | |
| Lactose | 70 mg | 700 | g |
| Calcium stearate | 2 mg | 20 | g |
| FD&C Blue #1 Lake | 0.03 mg | 300 | mg |

Trospium Chloride can be blended with lactose and cellulose until a uniform blend is formed. The lake can be added and further blended. Finally, the calcium stearate can be blended in, and the resulting mixture can be compressed into tablets using, for example, a 9/32 inch shallow concave punch. Tablets of other strengths may be prepared by altering the ration of active ingredient to the excipients or altering the final weight of the tablet. Slow-release or controlled-release tablets may be particularly valuable or useful.

Pharmacological Studies of Trospium

1. Ligand Binding Studies: Affinity for Muscarinic Receptors.

Methods:

The experiments were carried out on membranes prepared from SF9 cells that expressed human recombinant muscarinic receptor subtypes. After incubation with the test article and the proper radioligand (³H scopolamine methylchloride) and washing, bound radioactivity was determined with a liquid scintillation counter, using a commercial scintillation cocktail. The specific radioligand binding to a muscarinic receptor was defined as the difference between total binding and nonspecific binding determined in the presence of an excess of unlabelled atropine. IC₅₀ values (concentrations required to inhibit 50% of specific binding) were determined by non-linear regression analysis of the competition curves, from which affinity (pKi) values were determined (Cheng Y. et al. Biochem Pharmacol 1073, 22: 3099-3108.)

Results:

Affinity (negative logarithm of the dissociation constant Ki) of trospium and reference compounds for human recombinant receptors

| Test compound | M-1 | M-2 | M-3 | M-4 |
|---|---|---|---|---|
| Trospium | 9.1 | 9.2 | 9.3 | 9.0 |
| Tolterodine | 8.8 | 8.1 | 8.7 | 7.9 |
| Oxybutynin | 8.8 | 7.9 | 8.8 | 8.2 |
| Darifenacin | 8.3 | 7.7 | 9.2 | 7.4 |

Conclusions:

Trospium had slightly higher affinity for human muscarinic receptors than the reference compounds. The therapeutic activity of anti-muscarinic drugs in overactive human bladders are usually considered to be related to affinity for M-2/M-3 receptors, while the side effect xerostomia (dry mouth) is due mainly, but not exclusively, to inhibition of M-1 receptors in salivary glands.

2. Functional Characterization of Antimuscarinic and Antispasmodic

Activities on Bladder Smooth Muscle Strips.

Methods:

Experiments have now been performed using methods described by Kachur et al, 1988, J Pharmacol Exp Ther 247: 867-872) and Noronha-Blob et al. J Pharmacol Exp Ther 256: 562-567). Strips of tissue (approximately 10 mm long and 1.5 mm wide) were removed from the urinary bladder of male guinea pigs. The tissues were suspended in an oxygenated buffer of the following composition, in mM: NaCl 133; KCl 4.7; CaCl₂ 2.5; MgSO₄ 0.6; NaH₂PO₄ 1.3; NaHCO₃ 16.3; and glucose 7.7. The smooth muscle strips were maintained at or about 37.5° C. in tissue chambers and allowed to equilibrate with the bathing solution for one hour before proceeding with the experiment. Contractions induced by carbachol were used to measure anticholinergic actions of trospium and reference compounds (oxybutynin, tolterodine) as described by Smith et al., (Smith E. R., Wright S. E., Aberg G., Fang Y., McCullough J. R. Arzneim.Forsch. 1998, 48: 1012-1018).
   Results:

The antimuscarinic compounds terodiline, tolterodine, oxybutynin and trospium, potently inhibited carbachol-induced contractions with K_{B} values between 1.5 nM and 5.5 nM. There were no differences of biological significance between these test compounds, thereby confirming results from receptor binding studies in this functional test system.

3. Cognitive Side Effects.

Test Protocol 1.

Methods:

Conscious rats are used. The animals are placed at the base of a T-shaped maze with a reward placed in one of the arms of the maze. The effect of the test article on learning is determined by investigating the time it takes in the maze before the rats choose the correct arm of the maze. Experiments were also performed in rats that had been pretreated with phenylephrine to increase the permeability of the blood-brain barrier. Control groups of animals were treated with placebo (vehicle only) or with oxybutynin.

Results:

Results from ongoing studies demonstrate that Trospium Chloride does not influence the learning behavior, while learning is delayed by oxybutynin. Results from ongoing studies also demonstrate that there is no further impairment of learning behavior when Trospium Chloride is administered to animals in which a learning disorder has been induced with oxybutynin. Pretreatment of the animals with phenylephrine did not uncover any impairment of learning behavior by trospium.

Discussion:

The present results demonstrate that learning behavior is impaired by administration of oxybutynin, but not by Trospium Chloride. In animal, in which impaired learning had been induced with oxybutynin, there is no further impairment or aggravation of learning by the administration of Trospium to the animals, even in animals were pretreated with phenylephrine, which is a drug that is known to increase the permeability of the blood-brain barrier. It is concluded that Trospium does not have unwanted side effects on learning in this model, when given to naive animals or when given to animals that already demonstrate cognitive impairment. As known by those skilled in the art of pharmacology or toxicology, this animal model of learning behavior in rodents is considered to be a relevant model for studies of effects of drugs on cognition in humans.

Test Protocol 2.

Methods:

Modified Irwin tests were performed. This is a routine neuro-pharmacological test method. Conscious mice were used. The procedure involved an initial phase of undisturbed observation and a later manipulative phase. An animal's assessment started by observing its undisturbed behavior in housing (body position, locomotor activity, etc.). Thereafter the animal was transferred by tail on the working table for the manipulative phase (spatial locomotion, gait, aggressiveness, etc.). The occurrence of any unexpected state was noted. The responses to test substances were assessed at 60 min, 120 min and 24 hrs after dosing. Animals were dose orally with trospium chloride, some groups of animals had been pretreated with phenylephrine to increase the permeability of the blood-brain barrier. Criteria included behavioral, neurological, autonomic and toxicity changes in comparison with vehicle-treated groups. The results were expressed for each group as the number of animals displaying behavioral changes versus the corresponding control group at each observation time. No statistical analysis was carried out since the lack of statistically significant differences were obvious to the investigator.

Results:

The test results demonstrate that trospium chloride did not influence the test results and animals treated with trospium behaved similar to the animals of the control group. Pretreatment of the animals was performed to increase the BBB permeation. There were still no significant effects of trospium on the tested parameters.

Discussion:

There is no single animal model for a cognitive disorder since humans is the only species believed to express cognition. In the present neuropharmacological test system no unexpected behavioral changes were observed in vehicle-treated animals. Trospium administered at three dose levels produced no noteworthy changes in the behavior in mice, indicating no effects by trospium on any of the neuropharmacological parameters. Pretreatment of the mice with phenylephrine caused minor basal changes in the behavior of mice, as can be expected from an adrenergic alpha-receptor agonist. There were no noteworthy differences in behavior between mice treated with phenylephrine and animals treated with phenylephrine and trospium. This indicates that trospium did not have central neuropharmacological effects, even under circumstances that allowed the drug an improved access to the brains of the test animals. Negative effects of oxybutynin on cognition in humans are well known (Donnellan C A, Fook L, McDonald P, Playfer J R. B M J 1997, 315: 1364; Katz I R, Sands L P, Biker W. J Am Geriatr Soc. 1998, 46:8-13) and validate the present animal model.

Equivalents

Those skilled in the art will recognize, or be able to ascertain, using no more than routine experimentation, many equivalents to the specific embodiments of the invention described herein. Such equivalents include the co-administration of at least one compound of the present invention with any other drug that is used to combat diseases such as smooth muscle hyperactivity or certain conditions, such as pain, in mammals, mentioned in this document. Such equivalents also include the co-administration of at least one compound of the present invention with any other compounds or drugs that may be used for urinary incontinence, or cognition disorders. Those skilled in the art of medicine will also realize that higher or lower doses than those indicated here may be preferred and the doses may be given more or less frequently than suggested here.

Those skilled in the art, will realize that smooth muscle motility disorders, in addition to urinary bladder disorders, include disorders of the gastrointestinal tract, including gastric reflux (heart burn), diarrhea and irritable bowel syndromes (IBS) and disorders of the urinary and ducts (e.g. “kidney stone pain”; urolithiasis) and the gall fluid ducts (e.g. “gall stone pains”; cholelithiasis and choledocholithiasis) and disorders of the smooth muscles of the airways (e.g. asthma, COPD and bronchitis).

The term “urge incontinence” is in this document synonymous with the term “over-active bladder” (OAB) and includes the disease called pollakiuria (frequent urinations).

Those skilled in the art of pharmacology, will realize that the compounds of the invention, being trospium and the active metabolites of trospium and possible salts and solvates thereof, having certain pharmacological properties, such as antimuscarinic activity on various receptor types, calcium antagonistic activity, spasmolytic activity on various types of smooth muscle etc., may be useful for other indications than those listed here.

1. A method for treating smooth muscle disorders in a human suffering from, or having a propensity for, a cognitive disorder, comprising administering to said individual a therapeutically effective amount of trospium or a pharmaceutically acceptable salt or solvate thereof or an active metabolite of trospium or a pharmaceutically acceptable prodrug, salt or solvate thereof, while avoiding drug-induced cognitive side effects or drug-induced worsening of existing cognitive disorders, such unwanted effects on cognition being commonly expected from antimuscarinic medication used for treating said smooth muscle disorders.

2. The method of claim 1, wherein said smooth muscle disorder is a voiding disorder.

3. The method of claim 2, wherein said voiding disorder is a urinary voiding disorder.

4. The method of claim 3, wherein said urinary voiding disorder is urinary urge incontinence.

5. The method of claim 1, wherein said metabolite is a spiroalcohol metabolite.

6. The method of claim 1, wherein the amount of trospium or the active metabolite thereof is administered from 1 mg to 240 mg per day.

7. The method of claim 1, wherein the amount of trospium or the active metabolite thereof is administered from 10 mg to 60 mg per day.

8. The method of claim 1, wherein the amount of trospium or a pharmaceutically acceptable salt or metabolite thereof is administered together with a pharmaceutically acceptable carrier.

9. The method of claim 1, wherein said cognitive disorder is a symptom of Cognitive Disorder NOS.

10. The method of claim 1, wherein said cognitive disorder is Aphasia.

11. The method of claim 1, wherein said cognitive disorder is a symptom of Apraxia.

12. The method of claim 1, wherein said cognitive disorder is a symptom of Agnosia.

13. The method of claim 1, wherein said cognitive disorder is a symptom of a symptom of Executive Dysfunction.

14. The method of claim 1, wherein said cognitive disorder is a symptom of Senile Dementia.

15. A method for treating smooth muscle disorders in a human, comprising determining whether said human suffers from, or has a propensity for, a cognitive disorder, and if said determination is positive, administering to said human in need thereof a therapeutically effective amount of trospium or an active metabolite thereof or a pharmaceutically acceptable salt thereof.

16. The method of claim 15, wherein said smooth muscle disorder is a voiding disorder.

17. The method of claim 16, wherein said voiding disorder is a urinary voiding disorder.

18. The method of claim 17, wherein said urinary voiding disorder is urinary urge incontinence.

19. The method of claim 15, wherein said propensity for cognitive disorder is expressed as on-and-off manifestations of a cognitive disorder.
